(19) United States
(12) Patent Application Publication
(10) Pub. No.: US 2013/0264288 A1
(43) Pub. Date: Oct. 10, 2013

(54) System and Method For Collecting And Treating Plasma Protein Fractions

(71) Applicant: TERUMO BCT, INC., Lakewood, CO (US)

(72) Inventors: Dennis J. HLAVINKA, Arvada, CO (US); Thomas J. FELT, Boulder, CO (US); Gabriela MALDONADO-CODINA, Longmont, CO (US)

(73) Assignee: Terumo BCT, Inc., Lakewood, CO (US)

(21) Appl. No.: 13/857,193

(22) Filed: Apr. 5, 2013

Related U.S. Application Data
(60) Provisional application No. 61/620,544, filed on Apr. 5, 2012.

(51) Int. Cl. A61M 1/38

(52) U.S. Cl. CPC A61M 1/38
USPC 210/650; 210/257.2; 210/202; 210/251; 210/195.2

(57) ABSTRACT

Method and apparatus for separating plasma from blood in a separation vessel, separating the separated plasma into desired plasma proteins in a plasma separator fluidly connected to the separation vessel to receive the separated plasma, and adding photosensitizer to the desired plasma proteins for viral inactivation treatment of such proteins.

### FIELD

This patent application relates to on-line pathogen reduction treatment of a separated plasma product.

### BACKGROUND

For transfusions of blood and blood components, whole blood from a single donor is typically separated into three components: plasma, red blood cells and platelets. Each component may be used to treat a multiplicity of specific conditions and disease states. For example, the red blood cell component may be used to treat anemia and replace red blood cell loss due to bleeding, the concentrated platelet component may be used to control bleeding, and the plasma component may be given to patients to increase blood volume, or may be separated off-line after collection into individual plasma proteins such as fibrinogen, von Willebrand factor, Factor VIII, Factor IX, Anti-thrombin III, Fibrin sealant, thrombin, Alpha I and IVIG. Plasma from multiple donors may also be collected and combined or pooled together, and the combined plasma pool fractionated into the desired plasma proteins.

The separation of the collected plasma component into various protein or plasma components or fractions is called plasma fractionation. Such fractionation is typically done by large scale fractionators which combine plasma from many donors and concentrate plasma proteins from the collected plasma by using the known techniques of cold alcohol fractionation (also known as Cohn fractionation) and chromatography.

There are traditionally two ways to obtain separated blood components from single donors. One way is to collect whole blood from donors and separate it into components some time period after the whole blood collection. Using this method, whole blood is collected into approved containers that are pyrogen-free and sterile, with sufficient anticoagulant for the quantity of blood to be collected. Whole blood which is collected in this way is separated into components in a lab by a technician, and separation typically occurs from between about 2 and 8 hours after collection in the United States, and between about 2 to 24 hours in Europe.

Another way to separate whole blood into components is by using an apheresis device. Such apheresis devices separate whole blood from a single donor connected on-line to the device into components automatically, and return any uncollected and unneeded blood components back to the donor during the collection procedure.

Apheresis devices may be used to separate the plasma component from the cellular components of a blood donation. Apheresis devices permit more frequent donations by a single donor due to the return of uncollected components. US Publication No.: US 2010-0042037 discloses separation of plasma proteins on-line or while connected to a donor using an apheresis system.

Pathogen reduction treatment may be used to reduce pathogens in whole blood or collected and separated blood products. Such treatments may include the addition of a photosensitizer and activation of such photosensitizer with light. One such treatment using riboflavin is described in U.S. Pat. No. 6,258,577.

### BRIEF SUMMARY

The embodiments relate to a method of collecting plasma fractions from whole blood comprising rotating a separation vessel; separating plasma from other blood components of the whole blood in the rotating separation vessel; providing separated plasma from the rotating separation vessel to a plasma separator; separating the plasma into at least one fraction including desired plasma proteins using the plasma separator; mixing the at least one fraction with photosensitizer; collecting the at least one fraction with photosensitizer after the mixing step.

Another aspect is an apheresis plasma separation system comprising: a rotor; a separation vessel mounted on the rotor for rotating therewith wherein blood is separated into plasma and other components in the separation vessel during rotation of the rotor; a plasma separator fluidly connected to the separation vessel to receive the separated plasma from the rotating separation vessel; the plasma separator comprising: a hollow fiber membrane wherein the hollow fiber membrane can separate at least some plasma proteins from the separated plasma; photosensitizer fluidly connected to the plasma separator; a collection container fluidly connected to the plasma separator for collecting the separated plasma proteins from the plasma separator with the photosensitizer.

A further embodiment is an integrated pre-connected disposable set for an apheresis system comprising: a removal/return assembly for removing and returning blood and blood components to a donor; a separation vessel fluidly connected to the removal/return assembly and adapted for mounting on a centrifuge for rotation wherein whole blood is separated in the separation vessel during rotation of the centrifuge into at least plasma and other components; a plasma collect assembly fluidly connected to the separation vessel to receive separated plasma comprising: a membrane plasma separator for separating the separated plasma into at least a plasma protein fraction; photosensitizer in the plasma collect assembly; and a collection container fluidly connected to the plasma separator to receive the plasma protein fraction and the photosensitizer.

### BRIEF DESCRIPTION OF THE DRAWINGS

FIG. 1 is a schematic view of an apheresis system including a plasma separation unit.

FIG. 2 is a schematic view of the blood separator/plasma separation tubing set for the system of FIG. 1.

FIG. 3 is a schematic view of the blood separator/plasma separation tubing set of FIG. 2 with optional dry cartridge.

FIG. 4 is a schematic view of a detail of the tubing set cassette of FIGS. 2 and 3.

FIG. 5 is a simplified schematic view of the plasma separation unit of FIG. 2 with optional pump location.

FIG. 6 is a simplified schematic view of the plasma separator unit of FIG. 3 with optional pump location.

FIG. 7 is a schematic detail of an alternative plasma protein collection assembly with prion filter for the tubing set of FIG. 2.

FIG. 8 is a schematic detail of an alternative plasma protein collection assembly with prion filter for the tubing set of FIG. 3.

FIG. 9 is a schematic view of the plasma protein collection assembly of FIG. 8 with on-line illumination.

FIG. 10 is a schematic detail of the tubing set of FIG. 8 showing an alternate location for the cartridge and prion filter.

FIG. 11 is a schematic view of an alternative blood separator/tubing set with additional plasma component collection.

FIG. 12 is a schematic view of an alternative blood separator/tubing set with plasma protein collection from separation from the extracapilary side of the plasma separator.

FIG. 13 is a schematic view of a bag of collected plasma proteins being treated for pathogen reduction.

FIG. 14 is a block diagram illustrating a process using pathogen reduction treatment and collected apheresis plasma proteins for further fractionation.

### DETAILED DESCRIPTION

It should be noted that like elements are represented using like numerals in all the figures and the description. One embodiment is described with reference to the TRIMA Accel® automated collection system (manufactured and sold by Terumo BCT, Inc., Lakewood, Colo., USA) but it should be noted that any apheresis system, such as, but not limited to the COBE® SPECTRA system, SPECTRA OPTIA® system and the TRIMA® automatic collection system all also manufactured and sold by Terumo BCT, Inc. may be used without departing from the spirit and scope of the invention.

The embodiments described herein may also may be used with the apheresis systems of other manufacturers such as the Autopheresis C system manufactured by Fenwal, Inc. Lake Zurich, Ill., U.S.A. or the PCS system as manufactured by Haemonetics Corp. of Bainbridge, Mass.

The embodiments may also use an endogenous photosensitizer, though other photosensitizers could be used. A “photosensitizer” is defined as any compound which absorbs radiation of one or more defined wavelengths and subsequently utilizes the absorbed energy to carry out a chemical process. Examples of such photosensitizers include porphyrins, psoralens, dyes such as neutral red, methylene blue, acridine, toluidines, flavine (acriflavine hydrochloride) and phenothiazine derivatives, coumarins, quinolones, quinones, and anthroquinones.

Also, endogenous photosensitizers may be used. The term “endogenous” means naturally found in a human or mammalian body, either as a result of synthesis by the body or because of ingestion as an essential foodstuff (e.g. vitamins) or formation of metabolites and/or byproducts in vivo. Examples of such endogenous photosensitizers are alloxazines such as 7,8-dimethyl-10-ribityl isoalloxazine (riboflavin), 7,8,10-trimethylisoalloxazine (lumiflavin), 7,8-dimethylalloxazine (lumichrome), isoalloxazine-adenine dinucleotide (flavine adenine dinucleotide [FAD]), alloxazine mononucleotide (also known as flavine mononucleotide [FMN] and riboflavine-5-phosphate), vitamin Ks, vitamin L, their metabolites and precursors, and napththoquinones, naphthalenes, naphthols and their derivatives having planar molecular conformations. The term “alloxazine” includes isoalloxazines.

The fluid containing the photosensitizer is exposed to photoradiation of the appropriate wavelength to activate the photosensitizer, using an amount of photoradiation sufficient to activate the photosensitizer, but less than that which would cause non-specific damage to the biological components or substantially interfere with biological activity of other proteins present in the fluid. The wavelength used will depend on the photosensitizer selected.

The activated photosensitizer inactivates any microorganisms contained in the fluid. As used herein, the term “inactivation of a microorganism” means totally or partially preventing the microorganism from replicating, either by killing the microorganism or otherwise interfering with its ability to reproduce.

One embodiment further includes an optional prion filter. The P-Capt® filter manufactured by Macopharma of Mouvaux, France removes prions from at least one blood component. This filter uses ligand technology attached to resin with filter media. Prion proteins attach to the ligands for removal from a blood component product.

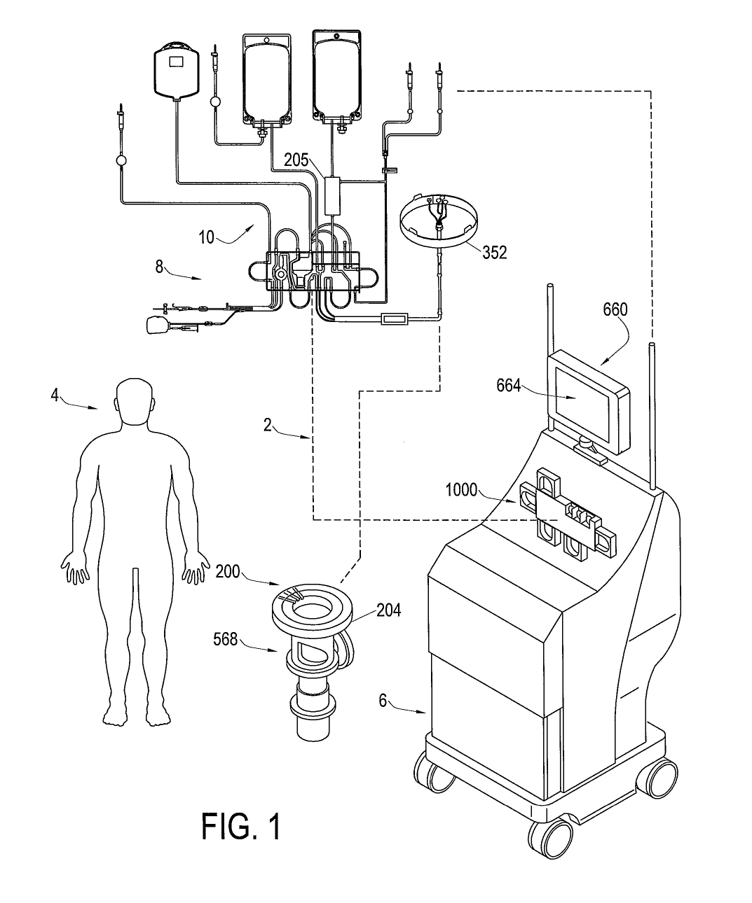

A blood apheresis system 2 is illustrated in FIG. 1 and allows for a continuous blood component separation process. Generally, in a continuous system or an on-line system, whole blood is withdrawn from a donor/patient 4 and provided to a blood component separation device 6 where the blood is separated into the individual blood components with at least one of these blood components being removed from the device 6 with the other components being returned to the donor. The continuous system 2 also provides for further separation or concentration of plasma into plasma proteins and the addition of photosensitizer to the plasma proteins for collection. Also, the continuous system may provide for filtration for removal of prions.

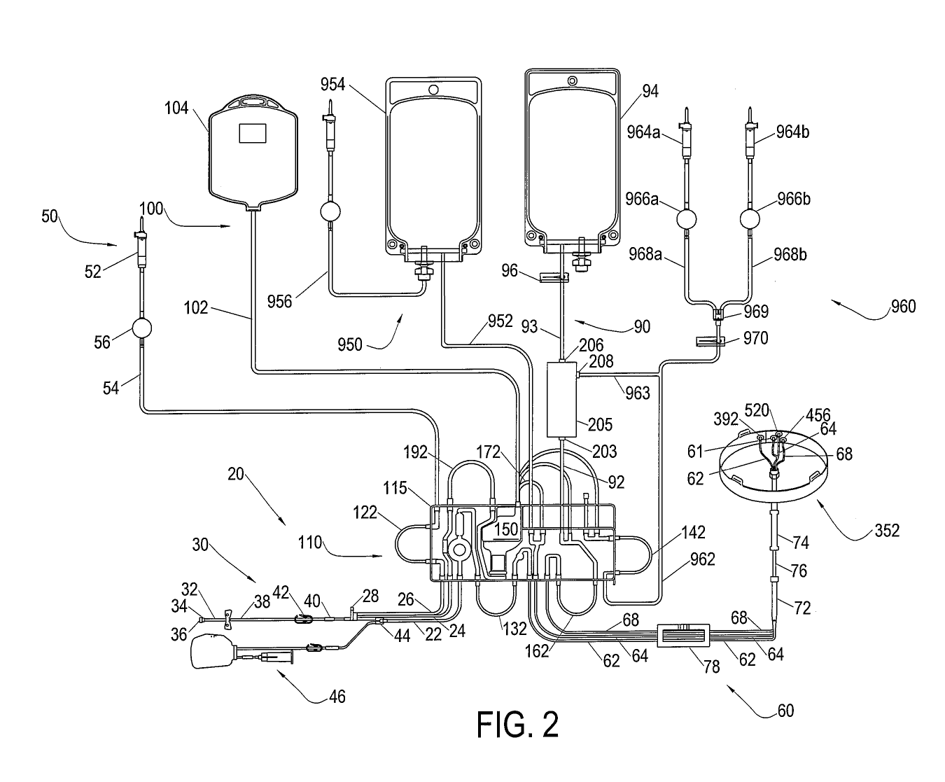

In the blood apheresis system 2, blood is withdrawn from the donor/patient 4 and directed through a pre-connected disposable set 8 (the disposable set embodiment of FIG. 2 is shown in FIG. 1) which includes an extracorporeal tubing circuit 10, a blood processing or separation vessel 352 and a plasma separator or concentrator 205 which defines a completely closed and sterile system. The disposable set 8 is mounted on the blood component separation device 6 which includes a pump/valve/sensor assembly 1000 for interfacing with the extracorporeal tubing circuit 10, and a channel assembly 200 for interfacing with the disposable blood processing vessel 352.

The channel assembly 200 includes a channel housing 204 which is rotatably interconnected with a rotatable centrifuge rotor assembly 568 which provides the centrifugal forces required to separate blood into its various blood component types by centrifugation. The blood processing vessel 352 is inter-fitted into the channel housing 204 to fit with a groove or channel in the channel housing. Blood thus flows from the donor/patient 4, through the extracorporeal tubing circuit 10, and into the rotating blood processing vessel 352. The blood within the blood processing vessel 352 is separated into various blood component types and at least one of these blood component types (e.g., plasma) is continually removed from the blood processing vessel 352. The plasma component may then be further concentrated or separated into plasma proteins to which photosensitizer is added as part of the continuous on-line system. Blood components which are not being retained for collection or for use in therapeutic treatments are also removed from the blood processing vessel 352 and returned to the donor/patient 4 via the extracorporeal tubing circuit 10. The blood processing vessel 352 may optionally be used for a platelet collection although such collection will not be described.

Operation of the blood component separation device 6 is controlled by one or more processors, not shown. In order to assist the operator of the apheresis system 2 with various aspects of its operation, the blood component separation device 6 includes a graphical interface 660 with a touch screen input/output device 664 connected to the processor.

The apheresis system below will be described with respect to a red blood cell collection and a plasma protein collection. Although an additional red blood cell collection is described it is further understood that other blood components, such as, but not limited to, platelets could alternatively, or, in addition, be collected. It is understood, however, that a plasma collection only may also occur if desired. If plasma collection only is desired, with subsequent separation and collection of plasma proteins, and addition of photosensitizer, the system described below can be simplified. For example, the red blood cell collection assembly 950 could be deleted. Also the replacement fluid assembly 960 can be optional. Collecting plasma proteins only with no red blood cell collection can provide a simplified closed system.

The apheresis system will also be disclosed with respect to pathogen reduction treatment of the desired plasma proteins to be collected and optional prion filtration.

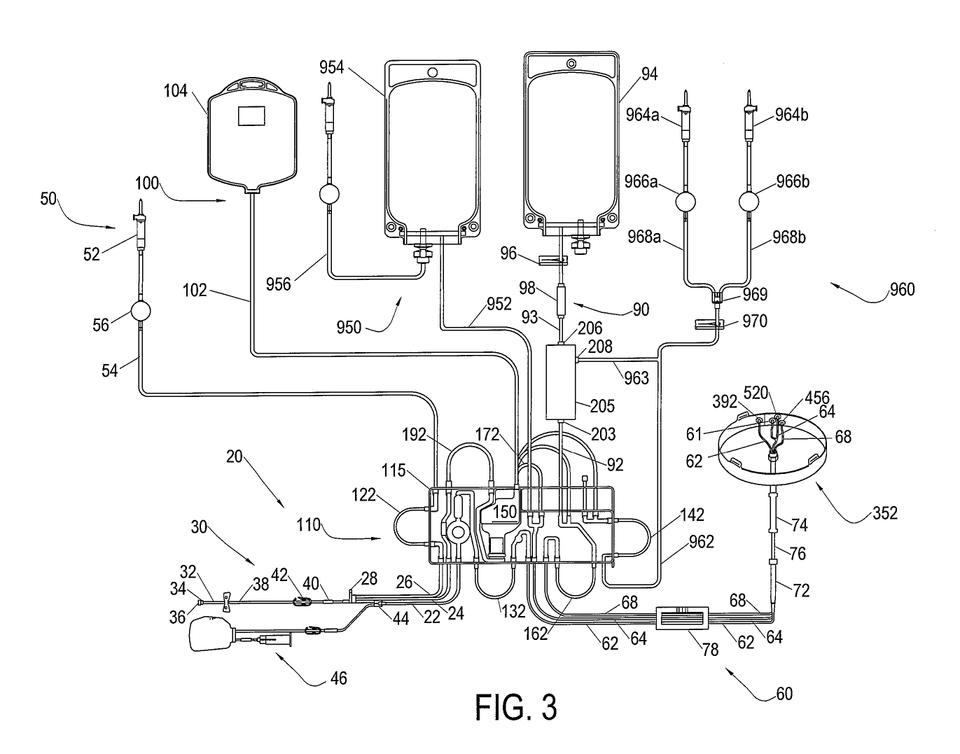

As illustrated in FIG. 2, and alternate embodiments FIGS. 3, 11 and 12, blood-primable pre-connected extracorporeal tubing circuit 10 comprises a cassette assembly 110 and a number of tubing assemblies 20, 50, 60, 950, 90, 100 and optionally 960 interconnected therewith. Generally, blood removal/return tubing assembly 20 provides a single needle interface between a donor/patient 4 and cassette assembly 110, and blood inlet/blood component tubing subassembly 60 provides the interface between cassette assembly 110 and blood processing vessel 352. An anticoagulant tubing assembly 50, plasma or plasma protein collection tubing assembly 90, red blood cell collection assembly 950 and vent bag tubing subassembly 100 are also interconnected with cassette assembly 110. Optionally, a replacement fluid sub-assembly 960 may be included. The extracorporeal tubing circuit 10 including the assemblies or sub-assemblies above and blood processing vessel 352 are interconnected to yield a closed disposable system or pre-connected disposable for a single use.

Cassette assembly 110 includes front and back molded plastic plates (not shown) that are hot-welded together to define a rectangular cassette member 115 having integral fluid passageways. The cassette assembly 110 further includes a number of outwardly extending tubing loops, described below, interconnecting various integral passageways. The integral passageways are also interconnected to the various tubing assemblies.

The blood removal/return tubing assembly 20 includes a needle subassembly 30 interconnected with blood removal tubing 22, blood return tubing 24 and anticoagulant tubing 26 via a common manifold 28. The needle subassembly 30 includes a needle 32 having a protective needle sleeve 34 and needle cap 36, and interconnect tubing 38 between needle 32 and manifold 28. Needle subassembly 30 further includes a D sleeve 40 and tubing clamp 42 positioned about the interconnect tubing 38. Blood removal tubing 22 may be provided with a Y-connector 44 interconnected with a blood sampling subassembly 46.

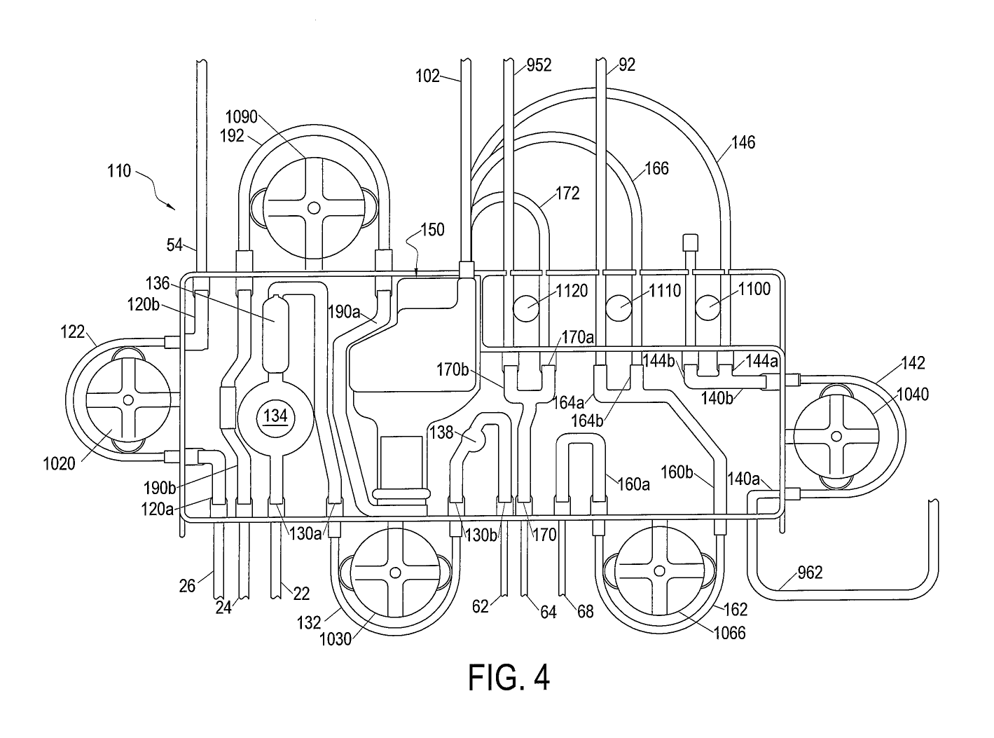

As shown in FIG. 4 the blood removal/return assembly includes first integral passageway 190a connected to the bottom of reservoir 150, tubing loop 192 and second integral fluid passageway 190b interconnected with tubing loop 192 and blood return tubing 24.

As seen in FIG. 4, cassette assembly 110 of FIGS. 2, 3, 11 and 12 includes a first integral anticoagulant passageway 120a interconnected with the anticoagulant tubing 26 of the blood removal/return tubing assembly 20. The cassette assembly 110 further includes a second integral anticoagulant passageway 120b and a pump-engaging, anticoagulant tubing loop 122 between the first and second integral anticoagulant passageways 120a, 120b. The second integral anticoagulant passageway 120b is interconnected with anticoagulant tubing 54. The anticoagulant tubing assembly 50 includes a spike drip chamber 52, (FIGS. 2, 3, 11 and 12) connectable to an anticoagulant source, anticoagulant feed tubing 54 and a sterile barrier filter 56. During use, the anticoagulant tubing assembly 50 supplies anticoagulant to the blood removed from a donor/patient 4 to reduce or prevent any clotting in the extracorporeal tubing circuit 10.

Cassette assembly 110 also includes a first integral blood inlet passageway 130a interconnected with blood removal tubing 22 of the blood removal/return tubing assembly 20. The cassette assembly 110 further includes a second integral blood inlet passageway 130b and a pump-engaging, blood inlet tubing loop 132 between the first and second integral blood inlet passageways 130a, 130b. The first integral blood inlet passageway 130a includes a first pressure-sensing module 134 and inlet filter 136, and the second integral blood inlet passageway 130b includes a second pressure-sensing module 138. The second integral blood inlet passageway 130b is interconnected with blood inlet tubing 62 of the blood inlet/blood component tubing assembly 60.

Blood inlet tubing 62 is also interconnected with input port 392 of blood processing vessel 352 to provide whole blood thereto for processing. To return separated blood components to cassette assembly 110, the blood inlet/blood component tubing assembly 60 further includes a red blood cell (RBC) outlet tubing 64 with outlet port 520 and plasma outlet tubing 68 with outlet port 456. Alternatively the outlet tubing and outlet ports could be for other blood components such as platelets. A control port for controlling the interface is shown at 61.

The blood inlet tubing 62, RBC outlet tubing 64, and plasma outlet tubing 68 all pass through first and second strain relief members 72 and 74 and a braided bearing member 76 there between. This advantageously allows for a sealess interconnection, as taught in U.S. Pat. No. 4,425,112 incorporated by reference herein. As shown, multi-lumen connectors 78 can be employed in the various tubing lines.

An optional replacement fluid tubing assembly 960 may be provided for delivery of replacement fluid such as sterile saline solution(s) (or replacement/exchange RBCs or plasma, e.g.) to the donor/patient 4. As shown, the replacement fluid assembly 960 includes at least a replacement fluid inlet tubing line 962 attached to the cassette 110 in fluid communication with an internal replacement fluid passageway 140a which is in turn connected to a replacement fluid tubing loop 142 which is connected back to the cassette 110 and an internal replacement fluid passageway 140b. Further internal passageways or spurs 144a and 144b and a tubing loop 146 are also shown. Internal passageway 144b is blocked off to disallow any fluid flow therein or therethrough. No outlet tubing line is preferably connected thereto and passageway 144b may also be omitted.

The replacement fluid assembly 960 further preferably includes one or more spike assemblies 964a-964b with optional associated sterile barrier devices 966a-966b and tubing connection lines 968a-968b which may be connected to tubing line 962 via a Y-connector 969 as shown. One or more slide clamp(s) 970 may also be included. As the plasma proteins may be frozen before use the sterile barrier devices 966a-966b are optional.

Although the replacement fluid assembly is shown as introducing such fluid through 140a and tubing loop 142 such is only exemplary. In other words the fluid could be introduced through other tubing loops for return such as tubing loop 162 or such fluid could even be aspirated through tubing into the system.

The plasma outlet tubing 68 of blood inlet/blood component tubing assembly 60 interconnects with a first integral plasma passageway 160a of cassette assembly 110. Cassette assembly 110 further includes a pump-engaging, plasma tubing loop 162 interconnecting the first integral plasma passageway 160a and a second integral plasma passageway 160b. The second integral plasma passageway 160b includes first and second spurs 164a and 164b. The first spur 164a is interconnected to the plasma collection tubing assembly 90. The plasma collection tubing assembly 90 may be employed to collect plasma proteins during use and includes plasma collector tubing 92, plasma separator or plasma separator unit 205, plasma collector tubing 93 and one or more plasma collection bags, containers or reservoirs 94. A slide clamp 96 may be provided on plasma collector tubing 93. The plasma collection tubing assembly 90 may also be employed for further separation of the plasma component as will be described in more detail below.

The second spur 164b of the second integral plasma passageway 160b is interconnected to a plasma return tubing loop 166 to return plasma to donor/patient 4. For such purpose, the plasma return tubing loop 166 is interconnected to the top of the blood return reservoir 150 of the cassette assembly 110.

The plasma return assembly also returns plasma after separation or concentration. The post separation return includes tubing 963 which connects to tubing 962, spurs 140a and 140b as well as pumps engaging plasma tubing loop 142. Spur 144a is connected to plasma return loop or tubing 146 to deliver plasma to cassette reservoir 150 for ultimate delivery to the donor/patient 4. Spur 140b is not used in the configuration shown and is closed off. Similarly this sub-assembly can be used to provide replacement fluid through 962, 140a, tubing loop 142, 140b and 146 to return reservoir 150. If no replacement fluid is required the portion of tubing 962 related to the replacement fluid sub-assembly above the connection with tubing 963 may be omitted.

Although the plasma return assembly is shown returning plasma through tubing loop 142, the plasma could also be returned through another pump loop arrangement such as 162.

The plasma collection tubing assembly further includes a plasma separation sub-assembly shown in FIGS. 2,3,5,6, 11 and 12 including hollow fiber membrane separator or concentrator 205. The hollow fiber membrane separator is the plasma separator or plasma separator unit. Tubing 92 is interconnected to the inlet 203 of the separator 205. Tubing 93 is interconnected to the outlet 206 of the membrane separator 205. Plasma collection tubing assembly 90 also includes tubing 963, interconnected to second outlet 208 for returning plasma or proteins that are not to be collected. The plasma return assembly including return tubing 963 connects to tubing 962 and spur 140a as described above.

The plasma protein membrane concentrator or plasma membrane separator 205 includes inlet 203 in first end cap 214 and outlet 206 in the opposite end cap 216.

Hollow fiber membranes are arranged between the two end caps 214 and 216. Such hollow fiber membranes include inter-capillary space (IC) within the hollow fibers and an extra-capillary space (EC) outside the hollow fibers. The pore size of the membrane forming the hollow fibers may be selected so that components such as plasma or optionally, protein of selected molecular weight may pass between the IC and EC spaces. Thus, if plasma separated from whole blood enters through tubing 92 and inlet 203 into the IC space, plasma and any proteins able to pass through the membrane pores to the EC space will pass through outlet 208 and tubing 963.

Table 1 below shows various protein factions and their molecular weight in kilodalton. The pore size of the membrane can be chosen to have a cut off value to pass through the membrane all but the desired protein fractions such as those given in the table. For example, the pore size could be such to pass all below 50 kilodaltons or the pore size could be selected to pass through the membrane those in a range in which the cut off value is selected between 50 kDa and 1300 kDa. The pore size could be selected to have a cut off value even lower than 50 kilodaltons such that only plasma liquid and sodium chloride pass through the membrane with all plasma proteins being collected.

TABLE 1

| Constituent | Molecular Weight (kDa) |
|---|---|
| Cholesterol | 1,300 |
| IgM | 950 |
| Fibrinogen | 340 |
| Factor VIII | 100-340 |
| IgE | 190 |
| IgD | 175 |
| IgA | 160 |
| IgG | 150 |
| Haptogloblin | 100 |
| Albumin | 66 |
| A1Antitrysin | 54 |
| Factor VII | 50 |

For example, a membrane having a pore size such that only constituents with a molecular weight of less than 50 kDa will pass, all proteins in Table 1 will be collected through outlet 206 with only plasma less proteins being returned through outlet 208.

For another example, a membrane having a pore size such that only constituents having a molecular weight of less than 150 kDa will be returned, only a portion of the proteins with a molecular weight greater than 150 kDa will be collected. Plasma and other plasma proteins of less than 150 kDa molecular weight will pass through the membrane to outlet 208.

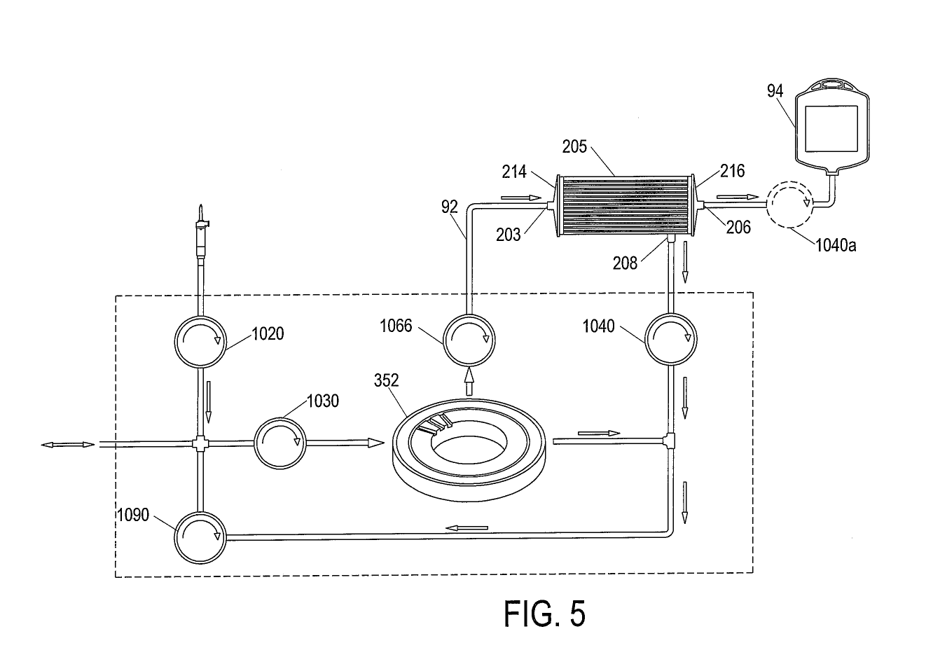

In the embodiment of FIGS. 2 and 5, plasma proteins to be collect are mixed with photosensitizer in bag or container 94. Outlet 206 of the membrane separator 205 is fluidly connected to the collection container or bag 94. Photosensitizer for pathogen reduction may be included in plasma collection bags, containers or reservoirs 94 as part of the tubing assembly and the plasma collection assembly. The pathogen reduction process will be more fully described below. The photosensitizer may be in liquid form in bag 94 or it, alternatively could be in dry form for mixing with the plasma proteins. The photosensitizer may be present in plasma collection bag 94 at any desired concentration from about 1 μM up to the solubility of the photosensitizer in the plasma proteins. For 7,8-dimethyl-10-ribityl isoalloxazine a concentration range between about 1 μM and about 160 μM may be used. The amount of photosensitizer to be mixed with the plasma proteins will be an amount sufficient to adequately inactivate microorganisms therein, but less than a toxic, (to humans or other mammals), or insoluble amount.

If the bag, container or reservoir 94 is the container used for illumination, it should be photo-permeable. Blood bag or photo-permeable container 94 may be prepackaged to contain the photosensitizer in the either dry or aqueous form as shown in FIGS. 2 and 5. The dry form may be in a dry powder form, a pill, capsule, tablet form or in various combinations therefore. The term dry solid or dry form envisions the components being in a loose powered state or in a solid state such as a pill, capsule, tablet capable of dissolving in fluid or any equivalent thereof known to one skilled in the art.

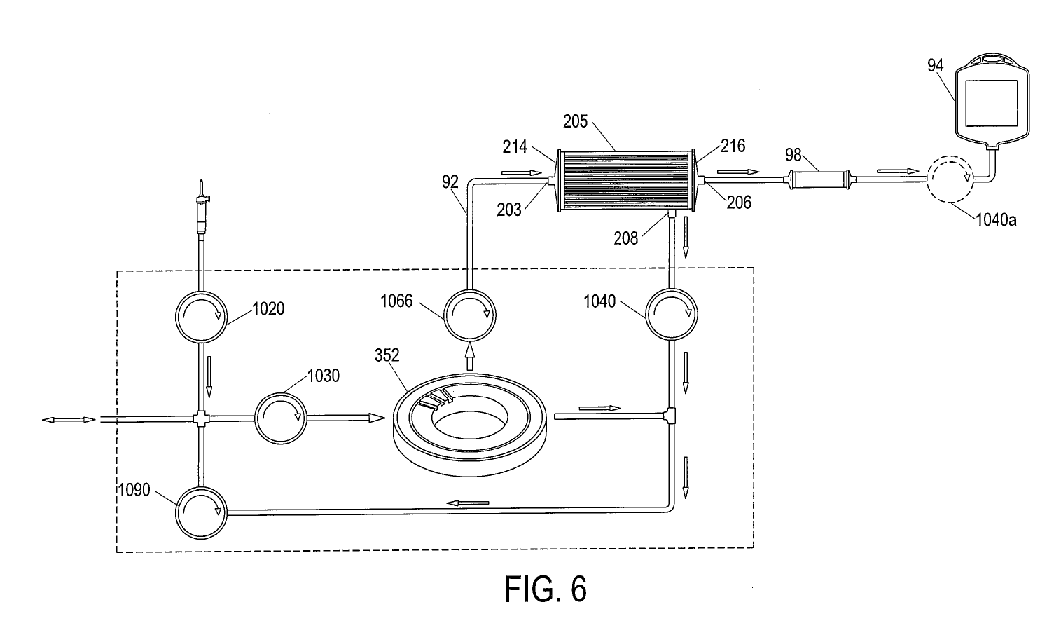

In the alternative embodiment of FIGS. 3 and 6, the photosensitizer is in dry form in cartridge 98 which is also part of the tubing assembly and the plasma collection assembly. The plasma proteins pass through cartridge 98, mixing with the dry photosensitizer, on the way to bag or container 94. The cartridge 98 could be another bag, a flask, a reservoir, a small cylinder or any similar container known in the art. Also the tubing 93 itself could contain certain forms of prepackaged photosensitizer. Cartridge 98 is located in plasma collector tubing 93 as shown in FIGS. 3 and 6. As described above the dry form may be in a dry powder form, a pill, capsule, tablet form or in various combinations therefore.

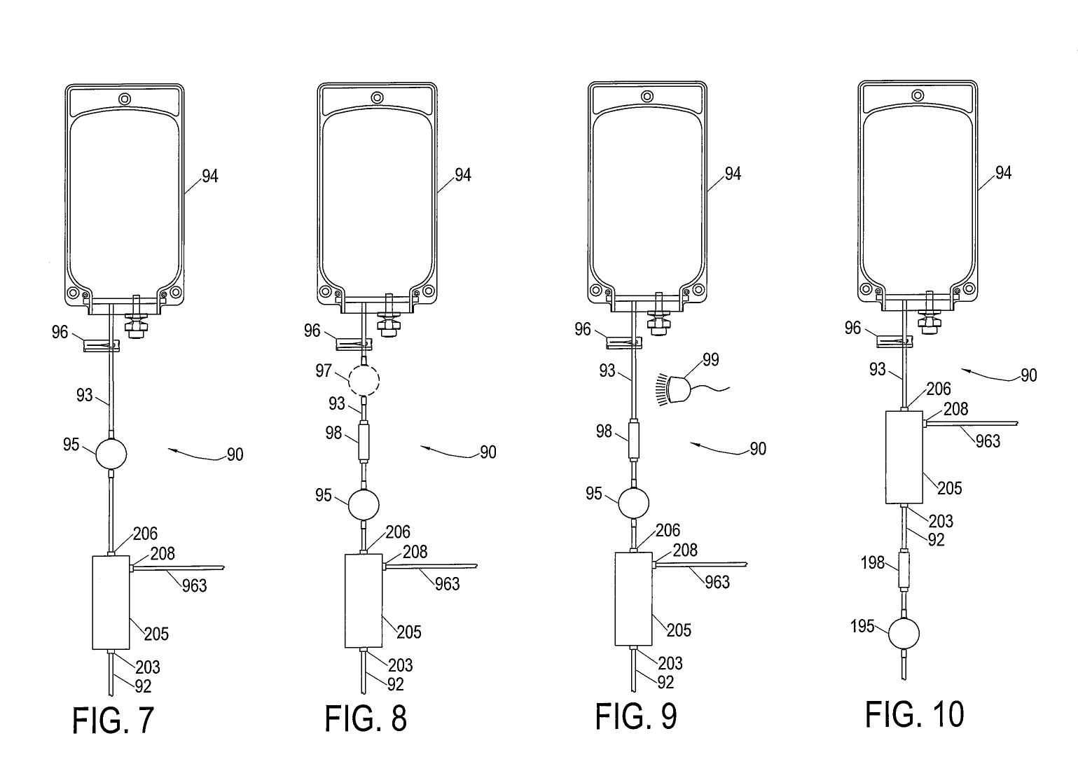

The plasma collection assembly 90 may also include an optional prion filter. Such a filter may be a media filter as shown or, alternatively could be a membrane filter. As shown in FIG. 7 the filter 95 may be located in tubing 93 between the outlet 206 of the membrane separator 205 and the collection bag 94. If the cartridge 98 is used to introduce dry form photosensitzer into the system and into bag 94, the filter 95 (shown in solid lines in FIG. 8), may be alternatively between the outlet 206 of the membrane separator and the dry cartridge 98 in tubing 93. Alternatively the filter may be between the cartridge 98 and the collection bag 94 in tubing 93 as shown in dashed lines in FIG. 8.

As shown in FIGS. 2, 3, and 4 the RBC outlet tubing 64 of the blood inlet/blood component tubing assembly 60 is interconnected with integral RBC passageway 170 of cassette assembly 110 (FIG. 4). The integral RBC passageway 170 includes first and second spurs 170a and 170b, respectively. The first spur 170a is interconnected with RBC return tubing loop 172 to return separated RBC to a donor/patient 4. For such purpose, the RBC return tubing loop 172 is interconnected to the top of blood return reservoir 150 of the cassette assembly 110. The second spur 170b may be closed off if red blood cells are not to be collected or may be connected with an RBC collection tubing assembly 950.

RBC collection tubing assembly 950 includes RBC collector tubing 952, at least one RBC collection reservoir, container, or bag 954, and sterile barrier filter/drip spike assembly 956. One or a larger practical number (not shown) of RBC bag(s) 954 may be connected to the collector tubing 952. Moreover, although not shown here one or more white blood cell (WBC) filtration devices and/or RBC storage solution connections and/or bags may also be pre-connected to and/or be included as component parts of the RBC collection tubing assembly 950.

Vent bag tubing assembly 100 is also interconnected to the top of blood return reservoir 150 of cassette assembly 110. The vent bag tubing assembly 100 includes vent tubing 102 and a vent bag 104. During use, sterile air present since packaging within cassette assembly 110, and particularly within blood return reservoir 150, cyclically passes into and back out of vent tubing 102 and vent bag 104, as will be further described.

As illustrated in FIG. 4, pump-engaging tubing loops 122, 132, 142, 162 and 192 extend from cassette member 115 to yield an asymmetric arrangement thereby facilitating proper mounting of cassette assembly 110 on blood component separation device 6 for use.

In normal operation, whole blood will pass through needle assembly 30, blood removal tubing 22, cassette assembly 110 and blood inlet tubing 62 to processing vessel 352. The whole blood will then be separated into blood components in vessel 352. During product collection, plasma, and optionally RBCs will be passed out of vessel 352 through corresponding ports 520 and 456 for collection or further separation. The plasma to be further separated will pass into the plasma separator 205 with photosensitizer being added to any collected plasma proteins.

In the cassette assembly the reservoir 150 having upper and lower ultrasonic sensors (not shown) is provided such that, during the blood processing mode, return blood will be removed from reservoir 150 during each blood return/replacement delivery sub-mode and accumulated during each blood removal sub-mode. When uncollected platelets and plasma (and potentially white blood cells) or red blood cells not collected and/or replacement fluid(s) have accumulated in reservoir 150 up to upper ultrasonic level sensor (not shown), operation of the pump 1090 associated with pump loop 192 will be initiated to remove the blood or replacement components from reservoir 150 through 190a, 192, and 190b and transfer the same back to the donor/patient 4 via the return/delivery tubing 24 and needle assembly 20. When the fluid level in the reservoir 150 drops down to the level of the lower ultrasonic level sensor, the return/delivery peristaltic pump 1090 will automatically turn off reinitiating blood removal sub-mode. The cycle between blood removal and blood return/replacement delivery sub-modes will then continue until a predetermined amount of plasma, and RBCs or other collected blood components have been harvested or collected.

Pump 1040 is associated with tubing pump loop 142, pump 1066 is associated with tubing loop 162, pump 1030 is associated with tubing loop 132, pump 1020 is associated with tubing loop 122, and pump 1090 is associated with tubing loop 192 when the cassette 110 is mounted on pump/valve/sensor assembly 1000.

The channel assembly 200 includes a channel housing 204 which is disposed on the rotatable centrifuge rotor assembly 568 (FIG. 1) and which receives a disposable blood processing vessel 352.

The channel housing 204 provides a mounting for the blood processing vessel 352 such that the blood may be separated into the blood component types in a desired manner. In this regard, the channel housing 204 includes a generally concave channel (not shown) in which the blood processing vessel 352 is positioned.

The blood processing channel vessel 352 is disposed within the channel housing 204 such that blood can be provided to the blood processing vessel 352 during rotation of the channel housing 204, to be separated into its various blood component types by centrifugation, and to have various blood component types removed from the blood processing vessel 352 during rotation of the channel housing 204. In addition, the channel allows for a blood priming of the blood processing vessel 352 (i.e., using blood as the first liquid which is provided to the blood processing vessel 352 in an apheresis procedure).

The blood processing vessel 352 is disposed within the channel of the channel housing 204 for directly interfacing with and receiving a flow of blood in an apheresis procedure. Further details of the blood processing vessel and parts of the apheresis system are described in U.S. Pat. No. 6,514,189B1.

As shown in FIGS. 2, 3, 11 and 12, blood is introduced into the interior of the blood processing vessel 352 through a blood inlet port 392 from inlet tubing 62. The blood inlet port 392 extends into an interior portion of the blood processing vessel 352.

Blood which is provided to the blood processing vessel 352 by the blood inlet port 392 is separated into at least plasma and optionally RBCs under centrifugal forces upon rotation of centrifuge rotor assembly 568 at an rpm for separation.

Separated plasma exits the blood processing vessel through port 456 and tubing 68. Separated red blood cells exit the blood through port 520 and tubing 64.

The apheresis system includes various valve assemblies shown schematically at 1120, 1110, and 1100 in FIG. 4. These valves are part of the pump/valve/sensor assembly 1000.

The apheresis system described herein provides for continuous separation of plasma and optionally red blood cells (RBCs) and/or plasma with continuous plasma separation and photosensitizer mixing. Both the plasma separation and photosensitizer mixing occurs on-line when the blood removal/return assembly is on the rotor assembly 568 with respect to the apheresis system step. Prion filtration may also occur on-line. For example, continuous separation may be provided with contemporaneous collection of plasma proteins with photosensitizer and/or with collection of RBCs. It is anticipated that four to eight tranfusable dosage double plasma protein products may be collected in a single apheresis procedure from a single donor.

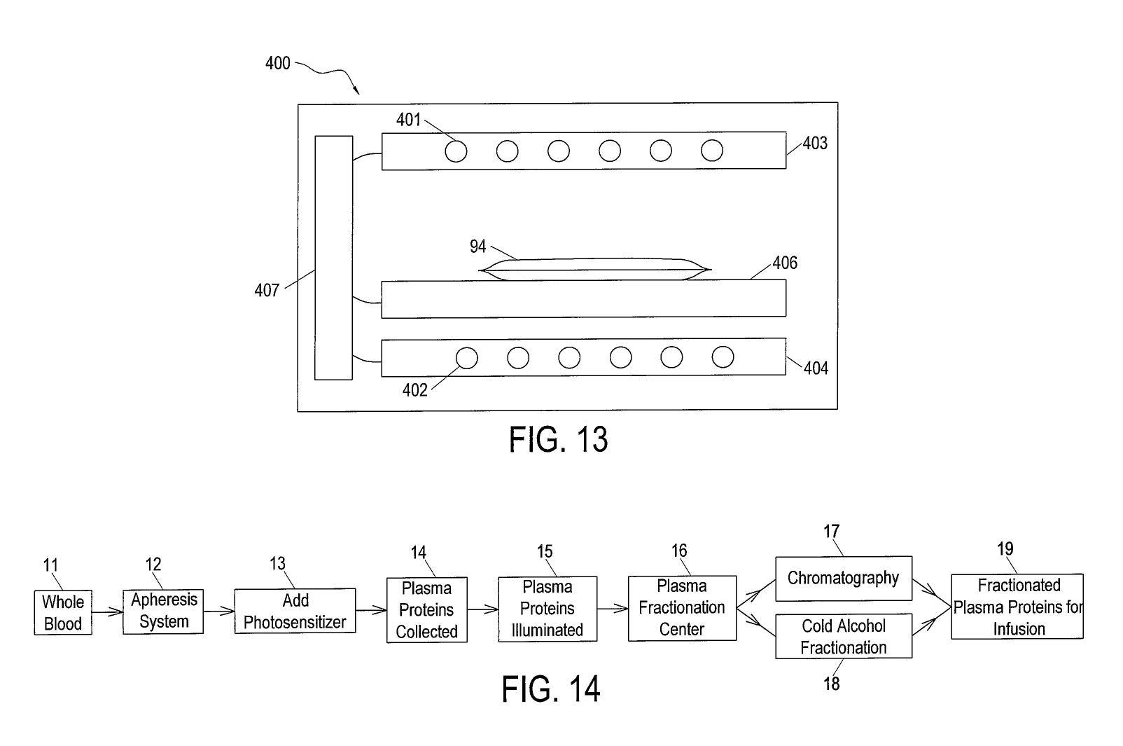

The plasma proteins to be treated may be illuminated in container and/or bag 94 or transferred to a photo-permeable container. The container to be illuminated is optionally agitated and exposed to photoradiation for a time sufficient to substantially inactivate the microorganisms. The photo-permeable container is made of transparent or semitransparent plastic, and the agitating device is preferably a shaker table. FIG. 13 illustrates a bag or container 94 under illumination. The illuminator is shown at 400. Bag 94 rests on support platform 406 which could be a shaker table. The radiation sources are shown at 403 and 404. The radiation emitting elements 401 and 402 may be visible or ultraviolet light or a combination thereof. The control unit 407 for the illuminator 400 controls the radiation sources as well as any shaker table.

Replacement fluid(s) are also optionally administrable within the procedures of the present embodiments. Sterile saline solution(s) is one of the optional replacement fluids considered for use herein. Thus, if/when large fluid amounts of plasma and/or RBCs are taken from a donor/patient, replacement fluid(s) may be delivered in return to leave the donor/patient adequately hydrated.

The initiation of blood processing provides for the collection of plasma product or plasma protein product in one or more reservoir(s) 94 containing photosensitizer optionally with collection of red blood cells in one or more reservoir(s) 954. Alternatively, either RBC collection in reservoir(s) 954 or plasma collection in reservoir(s) 94 may also be selectively completed in separate procedures. During either collection procedure, blood component separation device 6 preferably controls the initiation and termination of successive blood removal and blood return. Additionally, blood component separation device 6 will control the plasma and RBC collection processes according to predetermined protocols, preferably including control over the valve assemblies 1100, 1110 and 1120 of the pump/valve/sensor assembly 1000, and/or the appropriate pumps 1020, 1030, 1040, 1066 and/or 1090.

Initially, blood priming is carried out to prime the disposable system 10. During blood priming, it may be desirable that the component separation begins even during the priming stage, and that some plasma enters plasma protein collection tubing assembly 90. Thus plasma may flow out through the outlet port 456 to tubing 68.

Following and/or contemporaneously with the blood priming phase, blood separation control device 6 provides control signals to pump/valve/sensor assembly 1000 so that the optional replacement fluid lines may also be primed. In particular, replacement fluid valve assembly 1100 is opened and replacement fluid inlet pump 1040 is switched on to provide for the pumping of saline solution (or other replacement fluid(s)) through replacement fluid inlet tubing 962 and the replacement fluid tubing loop 142 into replacement fluid introduction tubing line 146 for initial collection in cassette reservoir 150, though this initial priming collection will likely and preferably does constitute a small amount of replacement fluid(s).

After priming is completed, yet still during the set-up phase, blood component separation device 6 may provide appropriate control signals to the pump/valve/sensor assembly 1000 such that all separated blood components flowing out of processing vessel 352 will first pass to return/delivery reservoir 150. Optionally, one or more cycles of separation and return of all blood components back to the donor may be performed before collection. Also, blood component separation device 6 may continue operation of blood inlet pump assembly 1030 associated with pump loop 132 during one or more these initial blood component return sub-modes.

To establish the desired AC ratio, blood component separation device 6 provides appropriate control signals to anticoagulant peristaltic pump 1020 so as to introduce anticoagulant into the blood inlet flow at a predetermined rate. The inlet flow rate of anti-coagulated blood to blood processing vessel 352 may be limited by a predetermined, maximum acceptable anticoagulant infusion rate (ACIR) to the donor/patient 4.

When collection begins, blood component separation device 6 may provide control signals so that plasma divert valve assembly 1110 switches to divert the flow of separated plasma pumped from vessel 352 through plasma outlet tubing 68 and plasma tubing loop 162 into plasma collector tubing 92 and into inlet 203 of membrane separator 205. See also FIGS. 5 and 6 which show simplified views of the apheresis system. Additionally, if plasma is to be collected alone, red blood cells will continue to flow from vessel 352 through outlet tubing 64 through return tubing loop 172 and into blood return reservoir 150. However, if RBCs are to be collected, contemporaneously with plasma, then red blood cell valve 1120 switches to divert the flow of separated RBCs flowing from tubing 64 to and through spur 170b (of cassette 110) and into and through tubing line 952 to the one or more RBC collection reservoir(s) 954.

During any of the collection processes, one or more replacement fluid(s) may also be delivered to the donor/patient 4. Thus, whenever the separation device 6 is in a collection rather than the return mode, the replacement fluid inlet valve assembly 1100 may also be opened and the replacement fluid pump 1040 starts to flow replacement fluids from the fluid source (not shown) through tubing line 962, cassette passageways 140a and 140b, and tubing loops 142 and 146 into the reservoir 150.

During separation and collection, channel housing 204 can be typically driven at a rotational velocity of about 3000 rpms to achieve the desired hematocrit during the both the setup and component collection phases. Correspondingly, the blood inlet flow rate to vessel 352 may be established at below about 64.7 ml/min. The desired hematocrit can be reliably stabilized by passing about two whole blood volumes of vessel 352 through vessel 352 before the RBC and/or plasma collection phases are initiated.

With respect to plasma collection, which may occur separate from or continuously with red blood cell collection, the separated plasma is pumped via pump 1066 through the plasma collect line 92 through filter separator or concentrator 205 to plasma component collection bag 94 through line 93. The pore size of the filter 205 determines whether all proteins are collected in container 94 or only those proteins of sufficiently high molecular weight.

The separated plasma is pumped out of rotor 352 through port 456, line 68, passageway 160a, tubing 162, passageway 160b, by pump 1066 around which tubing 162 extends and flows via plasma collect line 92 into filter or separator 205. The fraction of plasma proteins that do not pass through the filter membrane from the IC to the EC side enter tubing 93 and flow into storage bag 94. In the embodiments of FIGS. 2 and 5, photosensitizer contained in plasma component collection bag 94 mixes in the bag 94 with the collected plasma component or plasma proteins. This photosensitizer may be in liquid form for ease of mixing though dry form may be used. In the embodiment of FIGS. 3 and 6, the photosensitizer, in dry form, is dissolved and mixed with the plasma component or plasma proteins as they pass through cartridge 98 in tubing 93 on their way to bag or container 94. Thus, the bag or container 94 will contain plasma proteins mixed with photosensitizer.

If a prion filter is included in tubing 93 as shown in FIGS. 7, 8 and 9 additional filtration of the desired plasma proteins will occur to remove the specific prion proteins. As the plasma proteins are pushed through the filter 95, (or optionally 97) by the continuous process the filter media or filter ligands will capture and remove the prion proteins prior to collection of the desired plasma proteins in bag 94.

The remainder of the plasma and/or proteins that pass to the EC side flow out of the filter 205 through outlet 208, tubing 963, 962, passageway 140a, tubing loop 142, passageway 140b, tubing 146, to reservoir 150 and back to the donor 4.

An enriched plasma product, which may contain several times the normal amount or an increased concentration of the desired protein, could be produced by simply processing more plasma through the filter, concentrator or separator 205.

Following collection of the desired quantity of red blood cells, (if any), the separation and collection of plasma proteins, and after blood separation device 6 has provided control signals to divert assemblies 1110 and 1120 so as to divert the respective separated plasma and separated RBC flows to reservoir 150, if further blood processing is not desired, rinse back procedures may then be completed. The plasma pump 1066 is set at the full plasma rate equal to rate of the return/delivery pump 1090 for rinse back.

At the end of the procedures, the plasma bag(s) 94 and the red blood cell reservoir(s), if any, 954 may be disconnected from the extracorporeal tubing circuit 10.

After disconnection of the plasma bag(s) 94, such bags may be placed in an illuminator 400 to activate the photosensitizer to inactivate any pathogens contained therein as shown in FIG. 13. Alternatively, the plasma proteins could be transferred to a photopermeable bag for illumination. Radiation sources 403, 404 illuminate bag 94 (or a subsequent bag containing the contents of bag 94) with optional agitation of such bag.

FIG. 9 indicates another option for photoradiation. In this embodiment the plasma proteins with photosensitizer are exposed to the required radiation to activate the photosensitizer on-line. As shown in FIG. 9, the photosensitizer is added to the plasma proteins to be collected when such proteins pass through the cartridge 98, (as described for the embodiment of FIGS. 3 and 6). However tubing 93 is made of sufficiently photo-transmissive material such that the needed radiation for photosensitizer activation can occur in the tubing 93. Radiation source 99 adds the required radiation. This embodiment illustrates a flow-through system which permits radiation while the fluid or plasma proteins flows by the source illuminator.

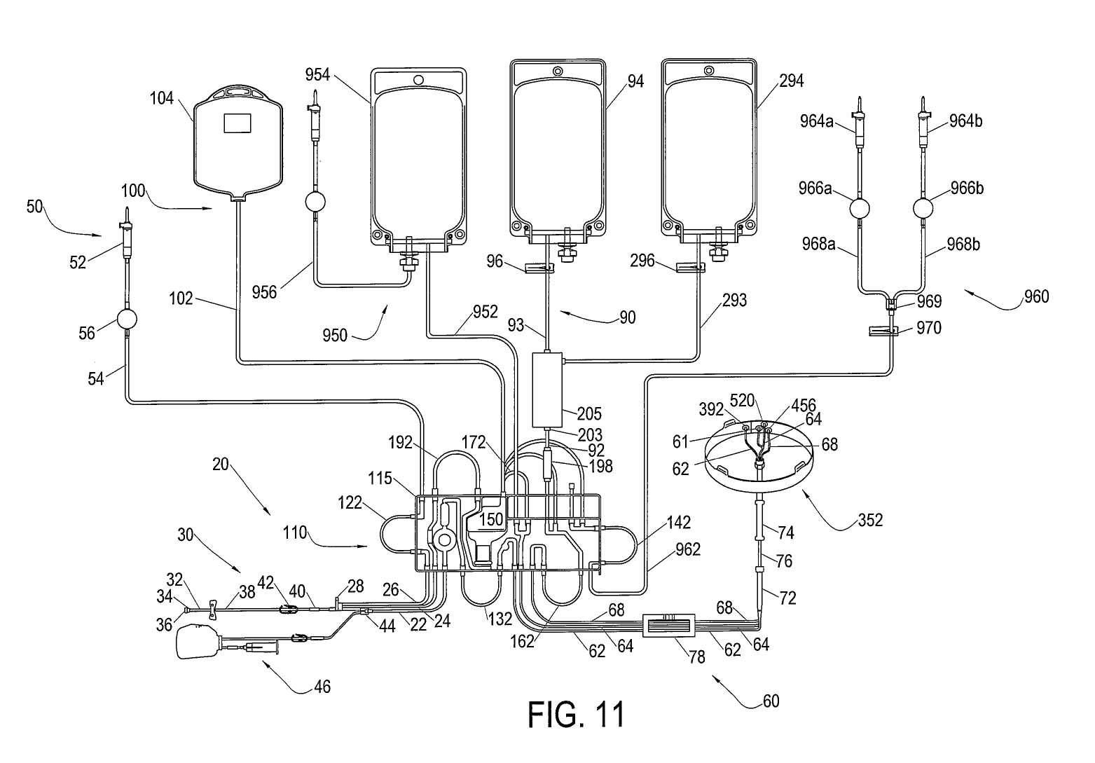

FIG. 10 illustrates an alternative plasma collection assembly. In this embodiment the separated plasma from the blood processing vessel 352 is filtered and mixed with photosensitizer prior to further plasma separation. As shown in FIG. 10, prion filter 195 is in tubing 92 to filter separated plasma from the blood processing vessel 352. After filtration, the filtered plasma mixes with photosensitizer as it passes through cartridge 198 containing dry form photosensitizer. This variation is particularly advantageous when both separated fractions of the plasma are collected as described below with respect to FIG. 11. Alternatively only the prion filter 195 or the cartridge 198 could be in tubing 92 with the other being in tubing 93. Cartridge 198 could also be omitted if the photosensitizer is in the plasma collection bag 94.

FIG. 11 illustrates an alternative embodiment wherein both separated fractions of plasma are collected. In this embodiment replacement fluid would typically be provided to the donor. As shown in FIG. 11 the cartridge 198 may provide the photosensitizer to be mixed with the plasma. The plasma, after separation in plasma separator 205, is provided to collection bags 94 and 294. As described previously plasma proteins that do not pass through the membrane of the membrane separator 205 may be collected in container or collection bag 94. Plasma liquid or ultrafiltrate as well as any proteins with molecular weights sufficiently low to pass through the membrane separator will pass through tubing 293 and open slide clamp 296 to second collection bag 294. If the cartridge 198 is not used, liquid or dry form photosensitizer may be in containers 94 and 294 for subsequent mixing with the plasma and proteins for collection.

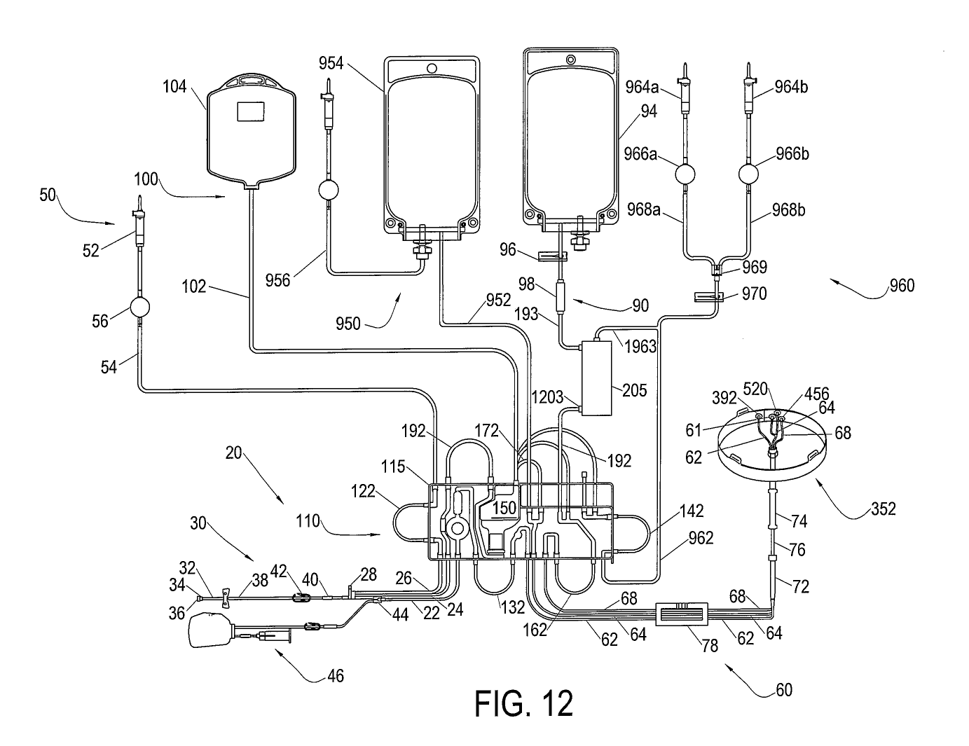

The embodiment of FIG. 12 illustrates a further separation tubing set wherein the separated plasma enters the EC side of the plasma separator 205 through inlet port 1203. Thus plasma proteins that do not pass through the membrane, in this variation may be collected by exiting the EC side passing through tubing 193 to plasma collection container 94. Although this embodiment shows plasma and plasma proteins that pass through the filter being returned to the donor from the IC side through tubing 1963 and 962 such low molecular weight elements could alternatively be collected in another collection container.

The simplified FIGS. 5 and 6 also indicate another option. As shown in FIGS. 5 and 6, the plasma entering the plasma separator 205 is pumped on the inlet side 203 by pump 1066 and also the outlet (EC) side 208 by pump 1040. However the locations of the pumps can be varied. For example, as shown in FIGS. 5 and 6 there also may be a pump on the IC exit side, (illustrated in phantom lines as 1040a). This pump may be used with the inlet pump 1066 alone, (no pumping through 1040 on the EC side) or it may be used with pump 1040 alone, (no pumping through 1066 into the inlet or IC side. Thus two pumps are utilized but the exact locations of such pumps may be varied.

Having pump 1040a pump on the IC side from 206 provides flow through the membrane by positive pressure on the IC side thus avoiding any degassing of the fluid as may occur using pumps 1066 and 1040 which exerts negative pressure on the EC side. If the membrane becomes blocked when pump 1040a is used the compression force of the rollers of pump 1040a could be such that they will lift sufficiently and provide less occlusion for the over volume or pressure. Thus it can function as a pressure relief valve.

This continuous apheresis procedure permits desirable proteins to be collected, with on-line preparation for subsequent pathogen treatment, and removed from a donor with the remainder of the plasma proteins being returned to the donor. This enables maximum collection and concentration of the desired proteins, instead of the smaller amount of desired protein contained within a single donation.

Using this procedure, plasma protein fractions with photosensitizer may be collected at the same time as other cellular components. Specifically, desired plasma proteins may be collected from a donor, while the undesired components may be returned to the donor. This would enable greater amounts of desired plasma proteins to be collected from a single donor, without increasing the risk to the donor as the amount of fluid volume removed from a donor would not be detrimental. More plasma can be processed resulting in the collection of increased amounts of plasma protein. However, from the donor perspective the increased collection of proteins can be collected with the same volume removal as a typical plasma collection.

The final concentration of the protein-enriched product could be adjusted by adjusting the ratio of the plasma flow into the filter and the plasma flow out of the filter. This can be done by adjusting pump speed of pump 1066, 1040 or 1040a. For example, if the membrane excluded all proteins and the flow rate through the filter was half that of the plasma flow into the filter, the resultant concentration of the proteins would be double that of normal donor plasma.

If it is desired to collect high molecular weight proteins, the filter/column 205 could separate on a continuous basis the albumin and other low molecular weight proteins and return them to the donor, while collecting higher molecular weight fractions such as fibrinogen, IgG, von Willebrand factor and factor VIII.

Alternatively, if lower molecular weight proteins are desired for collection, the higher molecular weight proteins could be returned to the donor, while the lower molecular weight proteins are collected by changing the tubing so that those that pass through the membrane are collected in a reservoir containing photosensitizer rather than returned. In this configuration outlet 208 would be connected to tubing 93 with outlet 206 being connected to tubing 963 for return to the donor.

The separation specificity can be accomplished by selecting membranes which have pore sizes which correspond to the molecular weight of the desired protein.

The high concentration protein collected could be used to enrich the plasma of a patient for therapeutic purposes. The high-concentration product may also be used for additional fractionation as described below where its yield of proteins would be much higher compared to normal plasma and thus produce an increased amount of protein concentrate products.

FIG. 14 illustrates, in block diagram form, the process of taking whole blood from a donor 11 (whole blood) and using apheresis apparatus 12 as described above to collect a concentrated protein fraction. The on-line process includes the addition of photosensitizer 13. The collected plasma proteins 14 are illuminated 15 to inactivate the photosensitizer. The collected plasma proteins from the apheresis process may optionally be provided to plasma fractionation center 16, and optionally pooled with other collections which have been treated for pathogen inactivation, for further fractionation or concentration of such product utilizing a known plasma fractionation process such as cold alcohol fractionation 18 (also known as Cohn fractionation) or chromatography 17. Other known fractionation processes could be used. This process could be used to provide a highly concentrated plasma protein infusion product 19 such as IVIG or clotting factor that has been treated for pathogen inactivation.

It will be apparent to those skilled in the art that various modifications and variations to the methods and structure of the present invention without departing from its scope. Thus it should be understood that the invention is not be limited to the specific examples given. Rather, the invention is intended to cover modifications and variations provided they come within the scope of the following claims and their equivalents.

1. An method of collecting plasma fractions from whole blood comprising:
rotating a separation vessel;
separating plasma from other blood components of the whole blood in the rotating separation vessel;
providing separated plasma from the rotating separation vessel to a plasma separator comprising a membrane wherein the membrane can separate at least some plasma proteins from the separated plasma;
separating the plasma into at least one fraction including desired plasma proteins using the plasma separator;
mixing the at least one fraction with photosensitizer;
collecting the at least one fraction with photosensitizer after the mixing step.

2. The method of claim 1 further comprising:
returning any plasma and plasma proteins not collected in the collecting step back to the donor.

3. The method of claim 1 further comprising:
separating red blood cells from other blood components in the rotating separation vessel;
collecting the separated red blood cells.

4. The method of claim 1 further comprising:
radiating with photo-radiation the at least one fraction and photosensitizer after the collecting step.

5. The method of claim 1 wherein the mixing step comprises mixing with 7,8-dimethyl-10-ribityl isoalloxazine as the photosensitizer.

6. The method of claim 1 further comprising:
providing the plasma separator comprising a hollow fiber membrane selected so that the separating the plasma step comprises:
passing plasma and any plasma proteins not to be collected through the membrane.

7. The method of claim 6 wherein the passing step comprises passing plasma and any low molecular weight plasma proteins through the membrane.

8. The method of claim 1 further comprising radiating with photoradiation the at least one fraction and photosensitizer prior to the collecting step.

9. The method of claim 2 wherein the returning step comprises pumping any plasma and plasma proteins not collected back to the donor.

10. The method of claim 2 further comprising:
separating red blood cells from other blood components in the rotating separation vessel;
collecting the separated red blood cells.

11. The method of claim 1 wherein the mixing step further comprises passing the at least one fraction through a cartridge containing photosensitizer in dry form to mix the at least one fraction with the photosensitizer prior to the collection step.

12. The method of claim 1 wherein the collecting step comprises collecting in a container.

13. The method of claim 12 wherein the mixing step comprises mixing in the container.

14. The method of claim 1 further comprising fractionating the at least one collected fraction in a plasma fractionation process to further concentrate the desired proteins in the at least one fraction.

15. The method of claim 14 wherein the step of fractionating the at least one collected fraction comprises precipitating out the desired proteins using cold alcohol fractionation.

16. The method of claim 1 further comprising filtering the at least one fraction to remove prion proteins.

17. The method of claim 1 further comprising:
separating the plasma into a second fraction;
collecting the second fraction.

18. The method of claim 1 wherein the mixing occurs prior to the separating step.

19. An apheresis plasma separation system comprising:
a rotor;
a separation vessel mounted on the rotor for rotating therewith wherein blood is separated into plasma and other components in the separation vessel during rotation of the rotor;
a plasma separator fluidly connected to the separation vessel to receive the separated plasma from the rotating separation vessel;
the plasma separator comprising a membrane wherein the membrane can separate at least some plasma proteins from the separated plasma;
a collection container fluidly connected to the plasma separator for collecting the separated plasma proteins from the plasma separator.

20. The system of claim 19 wherein the other components include at least a red blood cell component and further comprising:
at least one red blood cell collection container wherein any separated red blood components are collected in the red blood cell collection container.

21. The system of claim 19 further comprising:
a plasma return assembly for returning uncollected plasma from the membrane plasma separator to the donor, said plasma return assembly comprising a tube interconnected to a second outlet on said membrane plasma separator.

22. The system of claim 22 further comprising a replacement fluid assembly fluidly connected to said plasma return assembly.

23. The system of claim 22 wherein the membrane separator comprises a hollow fiber membrane comprising an intercapillary space and an extracapillary space and wherein said inlet and said first outlet are in fluid communication with said intercapillary space and said second outlet is in fluid communication with said extracapillary space.

24. The system of claim 21 wherein the membrane has a pore size such that uncollected plasma and low molecular weight proteins pass through the membrane and from the plasma separator to the return assembly.

25. The system of claim 19 wherein a photosensitizer is in the collection container.

26. The system of claim 19 further comprising a cartridge between the plasma separator and the collection container and wherein the photosensitizer is in the cartridge.

27. The system of claim 26 wherein the photosensitizer is in dry form in the cartridge.

28. The system of claim 19 further comprising a pump for pumping separated plasma from the separation vessel to the plasma separator.

29. The system of claim 19 wherein the membrane separator comprises a hollow fiber membrane.

30. The system of claim 19 further comprising an illuminator for providing photoradiation to the separated plasma proteins and photosensitizer.

31. The system of claim 30 wherein the illuminator provides photoradiation to the separated plasma proteins and photosensitizer in the collection bag.

32. The system of claim 30 wherein the illuminator provides photoradiation to the separated plasma proteins and photosensitizer prior to collection in the collection container.

33. The system of claim 19 further comprising a prion filter between the plasma separator and the collection container to remove prions from the separated plasma proteins.

34. The system of claim 26 further comprising a prion filter between the plasma separator and the cartridge.

35. The system of claim 26 further comprising a prion filter between the cartridge and the collection container.

36. The system of claim 19 further comprising a second collection container for the other components of the separated plasma.

37. The system of claim 19 further comprising a cartridge containing the photosensitizer between the separation vessel and the plasma separator.

38. The system of claim 19 further comprising a prion filter between the separation vessel and the plasma separator.

39. An integrated pre-connected disposable set for an apheresis system comprising:
a removal/return assembly for removing and returning blood and blood components to a donor;
a separation vessel fluidly connected to the removal/return assembly and adapted for mounting on a centrifuge for rotation wherein whole blood is separated in the separation vessel during rotation of the centrifuge into at least plasma and other components;
a plasma collect assembly fluidly connected to the separation vessel to receive separated plasma comprising
a membrane plasma separator for separating the separated plasma into at least a plasma protein fraction, said membrane plasma separator having an inlet and a first outlet;
photosensitizer in the plasma collect assembly; and
a collection container fluidly connected to the plasma separator to receive the plasma protein fraction and the photosensitizer.

40. The disposable set of claim 39 further comprising:
a plasma return assembly for returning uncollected plasma from the membrane plasma separator to the donor, said plasma return assembly comprising a tube interconnected to a second outlet on said membrane plasma separator.

41. The disposable set of claim 40 further comprising a replacement fluid assembly fluidly connected to said plasma return assembly.

42. The disposable set of claim 39 wherein the membrane separator comprises a hollow fiber membrane comprising an intercapillary space and an extracapillary space and wherein said inlet and said first outlet are in fluid communication with said intercapillary space and said second outlet is in fluid communication with said extracapillary space.

43. The disposable set of claim 39 wherein the photosensitizer is located in the collection container.

44. The disposable set of claim 43 wherein the photosensitizer is in liquid form.

45. The disposable set of claim 39 further comprising:
a cartridge between the membrane plasma separator and the collection container wherein the cartridge contains the photosensitizer.

46. The disposable set of claim 45 wherein the photosensitizer in the cartridge is in dry form.

47. The disposable set of claim 39 further comprising:
a filter in the plasma collect assembly to filter prions from the separated plasma protein fraction.

48. The disposable set of claim 47 wherein the filter is between the membrane plasma separator and the collection container.

49. The disposable set of claim 45 further comprising:
a filter in the plasma collect assembly to filter prions from the separated plasma protein fraction wherein the filter is between the cartridge and the collection container.

50. The disposable set of claim 43 wherein the photosensitizer is in dry form.

51. The disposable set of claim 45 further comprising:
a filter in the plasma collect assembly to filter prions from the separated plasma protein fraction wherein the filter is between membrane plasma separator and the cartridge.

52. The disposable set of claim 39 further comprising a second collection container to receive the other separated components of the separated plasma.

53. The disposable set of claim 39 further comprising a cartridge between the membrane plasma separator and the separation vessel wherein the cartridge contains the photosensitizer.

54. The disposable set of claim 39 further comprising a prion filter between the plasma separator and the separation vessel.
